(19) United States
(12) Patent Application Publication
(10) Pub. No.: US 2023/0320336 A1
(43) Pub. Date: Oct. 12, 2023

(54) COMPOSITION FOR PRODUCING CO2, USE OF A COMPOSITION FOR PRODUCING CO2, AND METHOD FOR PRODUCING CO2

(71) Applicant: Biogents AG, Regensburg (DE)

(72) Inventors: Martin GEIER, Sulzbach-Rosenberg (DE); Andreas ROSE, Regensburg (DE); Scott W. GORDON, Regensburg (DE); Maximilian EPPLE, Regensburg (DE)

(21) Appl. No.: 18/010,719

(22) PCT Filed: Jul. 1, 2021
(86) PCT No.: PCT/EP2021/068132

§ 371 Date: Dec. 15, 2022

(30) Foreign Application Priority Data
Jul. 7, 2020 (EP) 20184435.4

(51) Int. Cl. A01M 1/02; A01M 1/06; A01N 59/04; C12F 3/02

(52) U.S. Cl. CPC A01M 1/023; A01M 1/06; A01N 59/04; C12F 3/02; C12P 1/02

(57) ABSTRACT

The invention relates to a composition for the production of CO₂, the use of a composition for the production of CO₂ for an insect trap, in particular for an insect trap for attracting blood-sucking insects and arthropods, and a method for the production of CO₂. The composition comprises a component a), a component b), and a component c). Component a) comprises at least a first yeast strain, which has a low tolerance of less than 100 g of alcohol per liter. Component b) comprises at least one second yeast strain, which has a high tolerance of greater than 100 g of alcohol per liter. Component c) comprises at least one nutrient source for the at least one first yeast strain and/or for the at least one second yeast strain, wherein component c) is being formed by a turbo yeast or by a yeast extract.

### CLAIM OF PRIORITY

The present application claims priority to International Application PCT/EP2021/068132 filed Jul. 1, 2021, which in turn claims priority to European Application EP 20184435.4 filed Jul. 7, 2020, which are incorporated by reference.

### FIELD OF THE INVENTION

The present invention relates to a composition for the production of CO₂, the use of a composition for the production of CO₂ and a method for the production of CO₂ according to the features of the independent claims.

### BACKGROUND OF THE INVENTION

Blood-sucking insects often respond to olfactory and/or visual stimuli in order to track down humans or animals. A particular problem is, that blood-sucking insects often transmit diseases, which is why attempts are made to keep these insects away from human dwellings and/or gatherings. This can be done either by relying on repulsion, for example, by using chemical insect repellents. Alternatively, various insect traps are used to capture blood-sucking arthropods. Such insect traps often work with olfactory attractants and/or optical attractants.

For example, it is known that a combination of carbon dioxide and lactic acid is capable of attracting a number of blood-sucking insect species. For example, the US patent specification with the publication number U.S. Pat. No. 4,907,366 A describes the use of a composition of lactic acid, carbon dioxide and water in combination with heat to attract mosquitoes.

For example, the use of industrial gas cylinders is known, in order to provide carbon dioxide for the use in insect traps. However, this is relatively expensive and technically very complex. For this reason, the use of microorganisms such as yeast is also known, which produce carbon dioxide from a corresponding substrate by metabolization, in particular by fermentation and/or by respiration.

The publication of the application WO 99/26471 A1 describes an insect trap, in which insect trap baker's yeast within a nutrient solution is used to produce a continuous release of carbon dioxide.

### SUMMARY OF THE INVENTION

The task of the invention is to produce carbon dioxide in a particularly simple, inexpensive and quick way, especially for use in or with insect traps.

The above task is solved by a composition for the production of CO₂, the use of this composition and a method for the production of CO₂, which comprise the features in the independent patent claims. Further advantageous embodiments are described by the dependent claims.

The composition according to the invention comprises:
a component a) comprising at least one first yeast strain, wherein the at least one first yeast strain has a low tolerance of less than 100 g of alcohol per liter,
a component b) comprising at least one second yeast strain, wherein the at least one second yeast strain has a high tolerance of greater than 100 g of alcohol per liter,
a component c) comprising at least one nutrient source for the at least one first yeast strain and/or for the at least one second yeast strain, wherein component c) is formed by a turbo yeast or by a yeast extract.

In particular, the at least one first yeast strain contained in component a) is optimized for the production of carbon dioxide, wherein the production of carbon dioxide by the first yeast strain is done in a preferably particularly uniform way.

For example, component a) is formed by baker's yeast, which is optimized, in particular, to produce a lot of carbon dioxide in the absence of air and within a short time. The yeast can be contained within the composition in a moist form, for example, as pressed fresh yeast, as liquid yeast or as active dry yeast. In addition to the yeast cells, commercially available baker's yeast always contains minerals such as potassium, phosphorus, magnesium, calcium, etc., which originate, in particular, from the nutritional medium in which the yeast cells were grown. In particular, CO₂ production by the baker's yeast starts essentially immediately after the baker's yeast is added to a carbohydrate solution and increases essentially continuously and uniformly in the presence of an excess of carbohydrate.

Furthermore, the at least one second yeast strain contained in component b) is optimized for the production of alcohol. Preferably, component b) comprises at least one so-called distiller's yeast, a brewer's yeast and/or a wine yeast, which yeast, in particular, has an alcohol tolerance of between 110 grams per liter and 120 grams per liter.

Preferably, component b) can be formed by a so-called turbo yeast. Such so-called turbo yeasts are marketed specifically for the brewing business. The term “turbo yeast” used here represents a collective term and designates a mixture of a yeast and a yeast nutrient. Therefore, the term “turbo yeast” does not refer to a certain yeast, but to a mixture of a yeast, for example, a pure culture yeast, and a complex yeast nutrient. For example, such a turbo yeast consists of 58% of a distiller's yeast, 20% carbamide, 16% phosphates, 3% sulfates, 2% carbonates, and 1% vitamins and trace elements.

The so-called turbo yeasts contain at least one yeast strain that tolerates at least 14% of alcohol, in some cases the turbo yeasts tolerate up to 20% of alcohol or even more. Turbo yeasts are optimized to produce a lot of alcohol in the end. However, when using turbo yeast alone, the production of carbon dioxide starts only in a delayed manner.

Furthermore, it is advantageous if the first yeast strain and/or the second yeast strain are osmotolerant and can tolerate relatively high initial concentrations of sugar. In this way, all the sugar serving as a carbohydrate source can be added directly at once and the yeasts do not have to be re-fed with carbohydrate several times in order to maintain the corresponding CO₂ production over a desired period of time.

Another preferred embodiment provides, that the first yeast strain and/or the second yeast strain can not only metabolize simple sugars such as fructose and glucose, but in particular can also metabolize ordinary, cheap household sugar with the same rapidity. Another alternative embodiment provides, that the first yeast strain and/or the second yeast strain can also rapidly and effectively metabolize other carbohydrate sources such as flour or the like, for example malt flour, cereal flour, corn flour or flour from other suitable starch sources such as tapioca, yams, cassava or the like.

According to a preferred embodiment, component c) is formed by a turbo yeast. As described above, commercially available turbo yeasts comprise, in addition to an alcohol tolerant yeast strain, at least one nutrient source for the yeast strain. Thus, one embodiment of the composition may provide that the composition is formed from a first portion of baker's yeast and a second portion of turbo yeast. For example, equal parts of baker's yeast and turbo yeast may be used. Depending on the desired amount of carbon dioxide and the desired production profile over time, the composition may advantageously contain more baker's yeast or more turbo yeast.

According to a preferred embodiment, component c) is formed by a yeast extract. Yeast extract refers to a concentrate of the soluble ingredients of yeast cells. Yeast extract contains a high proportion of proteins, amino acids, nucleotides and vitamins of the B group. Yeast extract is produced in particular from predominantly protein-rich, specially bred yeast breeds (pure-bred yeast). These yeasts are broken down, which releases the cell juice. The yeast's own proteases and hydrolases then hydrolyze the cell contents, thereby splitting proteins into peptides and amino acids, and splitting DNA and RNA into nucleotides. To achieve a higher nucleotide content, the yeast's own enzymes can be supplemented by adding nucleases. The initially liquid autolysate or hydrolysate is subsequently liberated from the insoluble cell components, filtered, concentrated and, if necessary, spray-dried so that within a pasty yeast extract a dry substance content of 70-80% and within a powder 95-97% of dry substance is achieved

In one embodiment, components a:b:c are each in a freeze-dried form, for example, the composition thus comprises commercially available freeze-dried baker's yeast, commercially available freeze-dried turbo yeast, and commercially available freeze-dried yeast extract.

According to one embodiment, the components a:b:c are present in the composition in an amount ratio or weight ratio of 1-10:1-10:1-10. In particular, an amount ratio or weight ratio of 2:1:1 or 1:2:1 or 1:1:2 is preferred. The indicated amount ratios especially apply when all three components are preferably used in freeze-dried form. Corresponding quantity ratios apply when the components are used in fresh form with a moisture content or when at least one component is used in a freeze-dried form and one component is used in a fresh form with a moisture content. If necessary, the amount ratios must be adjusted in order to take this into account accordingly.

The above-described composition can be advantageously used whenever CO₂ is required over a long period of time. Particularly preferable, the composition is used to produce CO₂ for use within an insect trap, especially with an insect trap for attracting blood-sucking insects and arthropods. In particular, the composition makes it possible to continuously produce carbon dioxide over a longer period of time. In addition, cumulatively higher amounts of CO₂ are produced than when the individual yeast strains are each used individually in corresponding amounts.

Furthermore, a method for the production of CO₂ is described. Hereby, an aqueous solution is prepared from a carbohydrate source and a composition described above. For example, simple sugars such as fructose and glucose, or alternatively more complex sugars such as sucrose, serve as the carbohydrate source. In particular, provision may be made for the use of ordinary, inexpensive household sugar, or other carbohydrate sources such as flour or the like, for example malt flour, cereal flour, corn flour or flour from other suitable starch sources such as tapioca, yams, cassava or the like.

The aqueous solution produced by the method is characterized by the fact that especially after an initial starter phase, carbon dioxide is continuously released over a period of at least 12 hours. In particular, significantly higher amounts of carbon dioxide are generated with this method than when the individual components a, b, c are used in a correspondingly adjusted dosage. Preferably, the duration of the starter phase is a maximum of one hour when using the composition according to the invention.

To produce CO₂, an aqueous solution is produced from a carbohydrate source and a composition described above. Such an aqueous solution continuously releases CO₂ over a period of several hours, in particular over a period of at least 12 hours. Particularly preferably, the amounts of carbohydrate source and composition are chosen in such a manner, that the mixture releases carbon dioxide continuously for at least 24 hours or even for at least 48 hours. A further preferred mixture of carbohydrate source and composition according to the invention in aqueous solution is suitable for achieving a continuous, and after completion of an initial starter phase, a continuous and preferably uniform release of carbon dioxide over one to two weeks.

For example, the carbohydrate source can be formed by a monosaccharide, especially by glucose or fructose. Particularly preferably, the carbohydrate source is formed by a disaccharide, in particular by sucrose. In particular, conventional household sugar can be used, which makes it possible to produce CO₂ in an especially inexpensive manner.

According to one embodiment, water of a temperature between 30 and 40 degrees Celsius is used to produce the aqueous solution, in particular water of a temperature between 33 and 38 degrees Celsius, especially preferably water of a temperature of 35 degrees Celsius. This represents an optimal temperature for the yeasts and results in a quick start of the carbon dioxide production.

A particularly preferred embodiment provides that in order to prepare the aqueous solution, 500 grams of sugar and 20 grams of the composition are mixed in 2 liters of water, which water preferably has a temperature of 35 degrees Celsius. Preferably, the composition is thereby formed by 10 grams of component a); 5 grams of component b) and 5 grams of component c). Particularly preferred is a composition of 10 grams of commercially available baker's yeast, 5 grams of commercially available turbo yeast and 5 grams of commercially available yeast extract.

Thus, with the aforementioned mixture of a carbohydrate source and the composition described above, it is possible to continuously and substantially uniformly produce CO₂ over a desired extended period of time. This is particularly advantageous for the use with insect traps in longer-term field studies, where insect traps are to be set up at a specific location, and where the insects caught in the insect trap are to be counted and analyzed after a defined period of time. Hereby, it is important that the attractants of the insect trap are present in a preferably unchanging quantity over the entire experimental period, which can be ensured by the production of the carbon dioxide using the composition described within the context of the application.

The described composition can advantageously be used with both active insect traps and passive insect traps. Active insect traps refer to insect traps, for example, which insect traps are equipped with a fan, in which insect traps the attracted insects are sucked into a collection bag. Passive insect traps are, for example, those in which the attracted insects remain attached to an adhesive surface or similar or end up in catching containers, from which catching containers they cannot escape.

For the production of the carbon dioxide by using the composition, the corresponding aqueous solution can be prepared in any suitable container, which container can be arranged adjacent to the insect trap and comprises, for example, an outlet, via which outlet the carbon dioxide can be introduced specifically onto or into the insect trap. For example, it can be provided, that a connecting tube is attached to the container outlet in order to release the carbon dioxide at a defined place at or within the insect trap.

One embodiment may provide for the use of the composition to produce carbon dioxide in a special reaction bag. Such a reaction bag is preferably made of a thin plastic material and further comprises a closure assembly including a lid. The lid may preferably include a receiving or an attachment device for a connecting tube. Via the connecting tube, the carbon dioxide generated in the reaction bag is supplied to an insect trap.

For example, such insect traps are advantageously used in studies to capture flying insects and/or pest insects overnight for subsequent counting and/or categorization.

For example, it may be provided, that the carbon dioxide is introduced into the insect trap via the connecting tube and is subsequently discharged from the insect trap. Alternatively, the free end of the connecting tube can also be positioned adjacent to an insect trap so that the carbon dioxide flows out in the immediate vicinity of the insect trap and thereby serves to attract the insects.

Furthermore, it can be provided, that the reaction bag is equipped with a carrying handle in order to be able to transport it in a better and more easy way, especially when it is filled.

An aqueous mixture of a carbohydrate source and the composition according to the invention can be prepared directly by filling water, carbohydrate source and composition into the reaction bag. In this case, it may be provided, for example, that additionally a lid without a receiving opening is supplied in order to be able to shortly seal the reaction bag in a tight manner for mixing, following the filling of the components of the mixture into the bag. Alternatively, the components can also be mixed outside the reaction bag and the mixture is then filled into the same.

Preferably, it is provided, that the reaction bags are used several times. In particular, it can be repeatedly refilled with an appropriate mixture of water, carbohydrate and composition according to the invention.

Alternatively, it is possible to remove only part of the mixture from the reaction bag after a defined CO₂ production time has elapsed and add a new aqueous carbohydrate solution. The yeasts contained in the remaining mixture in the reaction bag provides a sufficient starter culture for further CO₂ production.

However, in this form, this is not possible as often as desired. Since the amount of nutrients provided by component c=the yeast extract decreases continuously, it may be necessary, in order to maintain the optimum conditions for CO₂ production, to further add yeast extract when the same starting composition is used several times.

The reaction bag can further be equipped with a temperature indicator, for example, a temperature measuring strip. In particular, this preferably comprises several temperature indicators so that the temperature on the surface of the reaction bag can be measured, whereby corresponding conclusions can be drawn about the temperatures prevailing inside the reaction bag. Preferably, the temperature indicators consist of substances that perform a reversible color change at certain temperatures, so that the reaction bags equipped with temperature indicators can preferably be used several times.

Furthermore, it may be provided, that the reaction bag is placed in a thermal container after filling with the appropriate mixture of water, carbohydrate source and composition in order to ensure an optimum reaction temperature and to prevent the effect of outside temperature on the CO₂ production.

The environment and thus the prevailing outside temperature has a strong influence on the CO₂ production by the yeasts. At temperatures above 38 degrees Celsius, especially at temperatures above 40 degrees Celsius, the yeasts die, which leads to a reduction in CO₂ production. It must be borne in mind here, that during CO₂ production heat is generated by the yeasts themselves, so that at high outside temperatures, such as those prevailing in tropical regions, these critical temperatures may easily be reached within the reaction mixture.

However, the arrangement of the reaction bag within a thermal container is also advantageous if there is a strong cooling down overnight, since the growth and thus the metabolism of the yeasts is significantly slowed down at cold temperatures, especially at temperatures below 30 degrees Celsius, which is reflected accordingly in the yields of carbon dioxide.

A further embodiment may provide that a bubble counter can be integrated into the connecting tube to measure the flow rate of the carbon dioxide. This can be particularly advantageous in the context of research projects in order to precisely analyze the experimental conditions.

With the composition according to the invention, an optimized CO₂ production in terms of continuity, quantity and uniformity of CO₂ release is achieved. By appropriately mixing at least two different yeast strains and a nutrient component, the respective optimized properties of the different yeast strains can be combined in a useful manner. Compared to the use of a pure first yeast strain, the composition comprising at least two different yeast strains is characterized by a higher CO₂ yield. Compared to the use of a pure second yeast strain, the composition is characterized, in particular, by the fact that the production of CO₂ starts significantly faster.

By appropriately adjusting the ratios of the composition and the carbohydrate source and/or by adjusting the ratios of the individual components a, b and c within the composition, in particular, a continuous and substantially uniform CO₂ production can be achieved over different desired time periods, in particular over time periods ranging from several days up to one to two weeks.

The composition can especially be produced at low costs because, for example, smaller quantities of the generally more expensive component b are contained in the mixture compared to the component that is generally cheaper to obtain. With the composition, the CO₂ can be produced much more cost-effectively compared to using corresponding quantities of dry ice or CO₂ compressed in a gas cylinder.

Another advantage is the easy disposal of the mixture. Since this is a mixture of water, alcohol and yeast, it can be easily disposed of without having to comply with any special regulations.

It should be expressly mentioned at this point that all aspects and embodiments which have been explained in connection with the device according to the invention equally concern or can be partial aspects of the method according to the invention. Therefore, if at any point in the description or also in the claim definitions relating to the device according to the invention reference is made to certain aspects and/or interrelationships and/or effects, this applies equally to the method according to the invention.

The same applies in reverse, so that all aspects and embodiments which have been explained in connection with the method according to the invention also equally concern or can be partial aspects of the device according to the invention. Therefore, if at any point in the description or also in the claim definitions relating to the method according to the invention reference is made to certain aspects and/or interrelationships and/or effects, this applies equally to the device according to the invention.

### BRIEF DESCRIPTION OF THE FIGURES

In the following passages, the attached figures further illustrate exemplary embodiments of the invention and their advantages. The size ratios of the individual elements in the figures do not necessarily reflect the real size ratios. It is to be understood that in some instances various aspects of the invention may be shown exaggerated or enlarged to facilitate an understanding of the invention.

FIG. 1 graphically shows the amounts of carbon dioxide produced by a composition according to the invention.

FIG. 2 shows a reaction bag for the production of carbon dioxide.

FIG. 3 shows the use of the composition within an insect trap.

### DETAILED DESCRIPTION OF THE INVENTION

The same or equivalent elements of the invention are designated by identical reference characters. Furthermore, and for the sake of clarity, only the reference characters relevant for describing the respective figure are provided. It should be understood, that the embodiments described are only examples describing an embodiment of the device and/or method according to the invention. They are not intended to limit the scope of the disclosure.

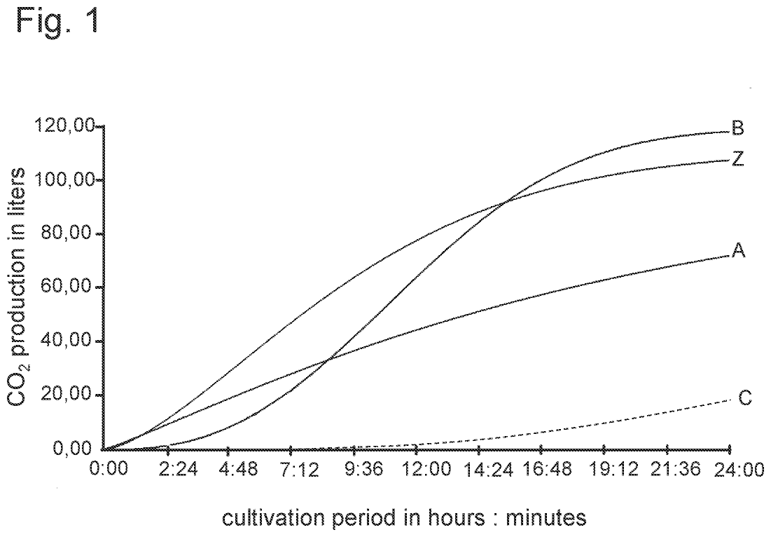

FIG. 1 graphically shows the amounts of carbon dioxide produced by a composition according to the invention. In the graph, the X-axis represents the cultivation period in hours and minutes. The Y-axis represents the CO₂ production in liters. In each case, an aqueous carbohydrate solution was prepared, which was mixed each with a defined amount of a single component a), b) or c) of the composition or with a defined amount of the composition. Subsequently, the CO₂ production in liters was measured over a cultivation period of 24 hours and the measured values were presented graphically.

Curve A shows the CO₂ production when using a defined amount X of an instant baker's yeast. Curve B shows the CO₂ production when using a defined amount X of a so-called turbo yeast. Curve C shows the CO₂ production using a defined amount X of a yeast extract and curve Z shows the CO₂ production using a defined amount X of a composition comprising 0.5 times instant baker's yeast; 0.25 times turbo yeast and 0.25 times yeast extract.

Curve A clearly shows that baker's yeast is especially optimized to produce large amounts of carbon dioxide in the absence of air and in a short period of time. In particular, CO₂ production in baker's yeast starts essentially immediately after the baker's yeast is mixed into the carbohydrate solution and increases essentially continuously and uniformly over the 24-hour period.

Turbo yeast is a mixture of a yeast and a yeast nutrient, which mixture is optimized for the production of alcohol and which is tolerating an alcohol content of at least 14%. For example, such turbo yeast consists of 58% of a distillers yeast, 20% of carbamide, 16% of phosphates, 3% of sulfates, 2% of carbonates, and 1% of vitamins and trace elements.

In curve B it can be seen, that the production of carbon dioxide starts with a delay; in particular, the CO₂ production only enters the linear range after about eight hours and saturation already occurs after a further eight hours. Saturation especially results in particular from the consumption of the carbohydrate source.

Furthermore, in curve C the CO₂ production is shown when using a yeast extract as component c). This is a dead yeast cell material, which is broken down to a greater or lesser extent depending on the manufacturer, but generally it contains no active yeast cells. The slight CO₂ production that begins after about eight hours can be explained by the fact, that the production of the carbohydrate solution and the addition of the yeast extract were not carried out under sterile conditions. Thus, organisms from the environment, for example from air, enter the nutrient solution formed by the yeast extract. The organisms multiply within the nutrient solution and also produce CO₂ through normal cellular respiration. The work was not carried out under sterile conditions, since when using the composition with insect traps, usually the work is not or cannot be carried out in a sterile manner either.

The comparison with curve Z showing the CO₂ production when using a composition of 0.5 parts instant baker's yeast, 0.25 parts turbo yeast and 0.25 parts yeast extract, reveals that CO₂ production using the composition starts immediately and already enters the linear range after about one hour. With the composition, significantly higher amounts of CO₂ can be produced continuously and essentially uniformly, especially within the first twelve hours compared to using only baker's yeast (curve A). Furthermore, CO₂ production within the first twelve hours is also optimized compared to the use of turbo yeast only (curve B), since in that case CO₂ production only starts with a delay.

For example, 500 g of household sugar are dissolved in two liters of warm water, in particular water at a temperature of about 35 degrees Celsius, and 20 grams of each of the individual components or 20 grams of the composition are added. With such a mixture, approximately 200 g of CO₂ can be generated over a period of 24 hours.

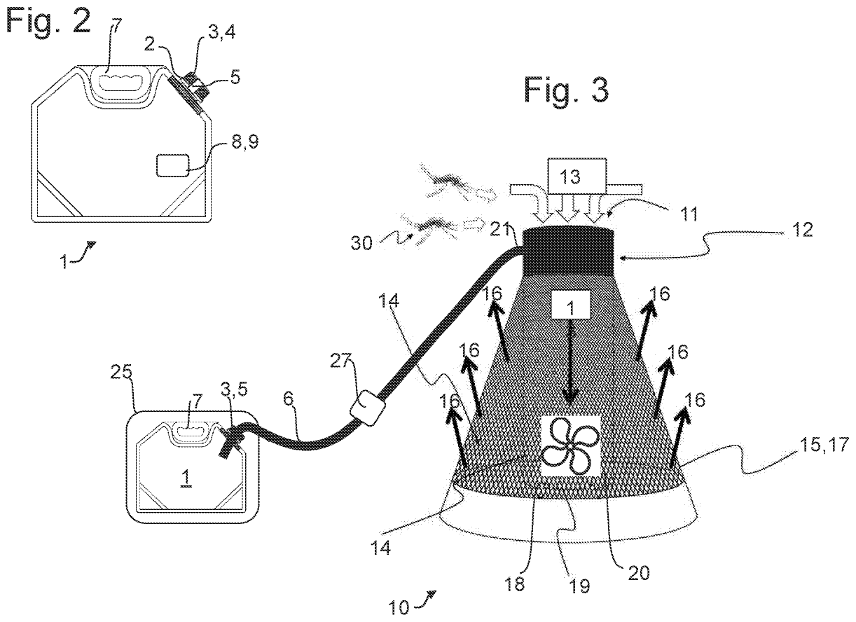

FIG. 2 shows a reaction bag 1 for the production of carbon dioxide by the composition described above. The reaction bag 1 preferably consists of a thin plastic material. The reaction bag 1 further comprises a closure arrangement 2 with a lid 3. Preferably, this is formed as a screw cap 4, which comprises a receiving opening 5 for a connecting tube 6 (see FIG. 3).

Furthermore, it can be provided, that the reaction bag 1 is equipped with a carrying handle 7 in order to be able to transport the reaction bag 1 better and more easily, in particular when it is in a filled state.

The aqueous mixture of a carbohydrate source, in particular sugar, and a composition according to the invention can be prepared directly therein by filling water, sugar and the composition into the reaction bag 1 and mixing them together therein. In this case, it may be provided, that additionally another lid without a receiving opening is provided, so that the reaction bag 1 can be sealed tightly for a short time after the filling in of the components of the mixture for mixing the same. Alternatively, the components can also be mixed outside the reaction bag 1 and the mixture is subsequently filled into the reaction bag 1.

Preferably, it is provided, that the reaction bag 1 can be used several times. In particular, the reaction bag 1 can be repeatedly refilled with an appropriate mixture of water, carbohydrate source and composition.

Alternatively, it is possible to remove only part of the mixture from the reaction bag 1 after a defined CO₂ production time has elapsed and add a new aqueous carbohydrate solution. The yeasts contained in the mixture remaining within the reaction bag 1 provide a sufficient starter culture for further CO₂ production. However, if necessary, this cannot be done as often as desired in this form, since the amount of nutrients, which is provided by component c), continuously decreases. If the same starter composition is used several times, it may therefore be necessary to add further yeast extract, in order to maintain the optimum conditions for CO₂ production.

The reaction bag 1 may further be equipped with a temperature indicator 8, for example with a temperature measuring strip 9. In particular, this temperature measuring strip 9 preferably has a plurality of temperature indicators in order to measure the temperature at the surface of the reaction bag 1, whereby corresponding conclusions can be drawn about the temperatures prevailing inside the reaction bag 1. Preferably, the temperature indicators consist of substances that perform a reversible color change at certain temperatures, so that the reaction bags 1 equipped with temperature indicator 8 can preferably be used several times.

FIG. 3 shows the use of the composition according to the invention for the production of carbon dioxide for an insect trap 10. In the embodiment shown, it is in particular an insect trap 10 for attracting and/or catching flying insects and/or pest insects 30 such as biting mosquitoes, mosquitoes, yellow fever mosquitoes, Zika mosquitoes, tiger mosquitoes or other blood-sucking insects, such as blood-sucking bugs, blood-sucking flies, fleas, lice, midges, sandflies, etc., etc., etc.

The insect trap 10 comprises an upper circular suction opening 11 continuing into a cylindrical suction channel 12, that is leading vertically downwards, in which suction channel 12 prevails an air flow pressurizing the suction opening 11 with negative pressure or with a suction flow 13 and leading to an interior 14 of the insect trap 10 or into it. In addition, the insect trap 10 is provided with a frustoconical outer wall 15, which outer wall 15 has a surface that is at least partially permeable to outflowing air 16, which outer wall 15 is formed, in particular, by a net-like structure 17, the mesh size of which is large enough for a sufficient outflowing air flow 16 to pass therethrough, but the mesh size of which reliably prevents escape of the insects 30 trapped within the interior 14 of the trap 10.

The outer wall 15 surrounds the suction channel 12 in the vicinity of the suction opening 11 and envelops the suction channel 12 in the further section continuing downwards at a varying radial distance, so that the outer wall 15 widens downwardly in a conical shape. In addition, the insect trap 10 is provided with a bottom side 18, which bottom side 18 adjoins the outer wall 15, is largely impermeable to incoming or outgoing air and is located opposite the suction opening 11, and which bottom side 18 is spaced from an open lower end side 19 of the suction channel 12, which extends into the interior 14 of the insect trap 10. In the illustrated embodiment of the insect trap 10, the bottom side 18 is planar and circularly shaped, and it is oriented perpendicular to the longitudinal axis of the suction channel 12.

At least one fan 20 generating the suction flow 13 may be arranged within the suction channel 12. The suction flow 13 has an air velocity which, if possible, makes it significantly more difficult for the attracted insects 30 to escape the vicinity of the suction opening 11. Rather, they are sucked into the interior 14 of the trap 10 with the aid of the sufficiently strong suction flow 13 and there they are reliably prevented from flying back out of the interior 14. Suitable means for restraining or killing the trapped insects 30, which are not shown here, may be arranged within the trap 10.

The insect trap 10 may be mounted in a hanging position or in a standing position in such a way that the suction opening 11 is directed upwardly and that the suction channel 12 preferably extends in an approximately vertical direction, and wherein the bottom side 18 forms a lower horizontal closure of the trap 10.

The outflowing air 16 already represents an attracting stimulus for the insects. As an additional attracting stimulus, the outflowing air is additionally enriched with carbon dioxide by introducing carbon dioxide into the insect trap 10 via a corresponding feed. This mixes within the insect trap 10 with the air drawn in by the suction flow 13 and flows out of the insect trap via the outer wall 15.

For example, a reaction bag 1 is filled with an aqueous solution comprising a sugar as a carbohydrate source and the composition according to the invention, which is described in the context of the application. A free end of a connecting tube 6 is inserted into the reaction bag 1 via the receiving opening 5 and attached to the reaction bag 1. The other free end of the connecting tube 6 is attached to a corresponding receiving opening 21 of the insect trap 10.

The carbon dioxide produced within the reaction bag 1 is introduced into the insect trap 10 through the connecting tube 6 and is discharged from the insect trap 10 again with the outflowing air 16 via the outer wall 15, by which it can exert its attracting effect on the flying insects and/or pest insects 30.

For example, such insect traps 10 are advantageously used as part of studies to capture flying insects and/or pest insects 30 overnight. The flying insects and/or pest insects 30 are subsequently counted and categorized as part of the study. The uniform production of carbon dioxide starts after approximately one hour. Thus, the preparation of the insect trap 10 should ideally begin approximately one hour before the start of the counting.

The reaction bag 1 can additionally be arranged in a thermal container 25. An optimum reaction temperature can be ensured within the thermal container 25. In particular, this prevents an effect of the outside temperature on the CO₂ production.

The environment and thus the prevailing outside temperature has a strong influence on the CO₂ production by the yeasts. At temperatures above 38 degrees Celsius, especially at temperatures above 40 degrees Celsius, the yeasts die, which leads to a reduction in the CO₂ production. It must be borne in mind here that heat is generated by the yeasts themselves during the CO₂ production, so that at high outside temperatures, such as those prevailing in tropical regions, these critical temperatures may well be reached within the reaction mixture even at night.

The arrangement of the reaction bag 1 within a thermal container 25 is also advantageous if, for example, it cools down considerably overnight, since the growth and thus the metabolism of the yeasts is significantly slowed down at cold temperatures, in particular at temperatures below 30 degrees Celsius, which is correspondingly reflected in a reduction in CO₂ production.

It may further be provided, that a bubble counter 27 may be integrated into the connecting tube 6 to measure the flow rate of carbon dioxide.

With the composition according to the invention, an optimized CO₂ production is achieved with regard to continuity, quantity and uniformity of CO₂ release. Through the appropriate combination of at least two different yeast strains and a nutrient component within the composition according to the invention, the respective optimized properties of the different yeast strains can be combined in a useful manner. Compared to the use of pure baker's yeast, the composition is characterized by a higher CO₂ yield. Compared to the use of pure turbo yeast, the composition is especially characterized by the fact that the production of CO₂ starts significantly faster and, in particular, passes significantly faster into the linear, uniform production range.

By appropriately adjusting the amount ratios of the composition in relation to the carbohydrate source and/or by adjusting the ratios of the individual components a), b) and c) within the composition, an especially continuous and substantially uniform CO₂ production can be achieved over different desired periods of time, in particular over periods of time ranging from several days up to one to two weeks.

The composition can be produced in an inexpensive manner because the composition contains, for example, smaller amounts of turbo yeast compared to the cheaper obtainable baker's yeast. With the composition, the CO₂ can be produced much more cost-effectively than when using corresponding quantities of dry ice or CO₂ compressed within a gas cylinder.

Another advantage is the easy disposal of the mixture. Since this is a mixture of water, alcohol and yeast, it can be easily disposed of without having to comply with any special regulations.

The embodiments, examples and variations of the preceding paragraphs, the claims or the following description and the figures, including their various views or respective individual features, may be used independently or in any combination. Features described in connection with one embodiment are applicable to all embodiments unless the features are incompatible.

Although the figures generally refer to “schematic” representations and views, this does not mean that the figure representations and their description are of secondary importance with respect to the disclosure of the invention. The person skilled in the art is quite capable of obtaining enough information from the schematically and abstractly drawn representations to facilitate his understanding of the invention without being impaired in any way in his understanding, for example, by the drawn and possibly not exactly to scale proportions of the device and/or parts of the device or other drawn elements. The figures thus enable the skilled person as a reader to derive a better understanding of the idea of the invention formulated in a more general and/or abstract manner in the claims as well as in the general part of the description on the basis of the more concretely explained implementations of the process according to the invention and the more concretely explained mode of operation of the device according to the invention.

The invention has been described with reference to preferred embodiments. To the expert it is also conceivable, however, to make changes and modifications without leaving the scope of protection of the appended claims.

### LIST OF REFERENCE SIGNS

1 reaction bag
2 closure arrangement
3 lid
4 screw cap
5 receiving opening
6 connecting tube
7 carrying handle
8 temperature indicator
9 temperature measuring strip
10 insect trap
11 suction opening
12 suction channel
13 suction flow
14 interior
15 outer wall
16 outflowing air
17 net-like structure
18 bottom side
19 lower end side
20 fan
21 receiving opening
25 thermal container
27 bubble counter
30 flying insects and/or pest insects
A CO₂ production when using instant baker's yeast
B CO₂ production when using turbo yeast
C CO₂ production when using yeast extract
Z CO₂ production when using the composition according to the invention.

We claim:

1.-13: (canceled)

14. A composition for the production of CO₂, comprising:
a component a) comprising at least one first yeast strain, wherein the at least one first yeast strain has a low tolerance of less than 100 g of alcohol per liter,
a component b) comprising at least one second yeast strain, wherein the at least one second yeast strain has a high tolerance of greater than 100 g of alcohol per liter, and
a component c) comprising a yeast extract or by a turbo yeast.

15. The composition of claim 14, wherein the at least one first yeast strain comprises a baker's yeast.

16. The composition of claim 14, wherein the at least one second yeast strain comprises a distiller's yeast, a brewer's yeast or a wine yeast.

17. The composition of claim 14, wherein the at least one second yeast strain comprises a turbo yeast.

18. The composition of claim 14, wherein components a), b), and c) are each in freeze-dried form.

19. The composition of claim 14, wherein components a), b), and c) are present in a weight ratio of 1-10:1-10:1-10.

20. The composition of claim 19, wherein components a), b), and c) are present in a ratio of 2:1:1 or 1:2:1 or 1:1:2.

21. The composition of claim 14, wherein the at least one first yeast strain or the at least one second yeast strain are osmotolerant and tolerate a high initial concentrations of sugar.

22. A method of attracting blood-sucking insects and arthropods, the method comprising producing CO₂ in an insect trap with a composition comprising:
a component a) comprising at least one first yeast strain, wherein the at least one first yeast strain has a low tolerance of less than 100 g of alcohol per liter,
a component b) comprising at least one second yeast strain, wherein the at least one second yeast strain has a high tolerance of greater than 100 g of alcohol per liter, and
a component c) comprising a yeast extract or by a turbo yeast.

23. The method of claim 22, wherein the producing CO₂ step continuously produces CO₂ for at least 12 hours.

24. The method of claim 23, wherein the producing CO₂ step continuously produces CO₂ for at least 24 hours.

25. A method for the production of CO₂, comprising making an aqueous solution of
a carbohydrate source; and
a composition comprising:
a component a) comprising at least one first yeast strain, wherein the at least one first yeast strain has a low tolerance of less than 100 g of alcohol per liter,
a component b) comprising at least one second yeast strain, wherein the at least one second yeast strain has a high tolerance of greater than 100 g of alcohol per liter,
a component c) comprising a yeast extract or by a turbo yeast,

26. The method of claim 25, wherein the carbohydrate source comprises a monosaccharide or a disaccharide, or a flour or any other suitable starch source.

27. The method of claim 26, wherein carbohydrate comprises glucose, fructose, or sucrose.

28. The method of claim 26, wherein the water temperature is between 33 and 38 degrees Celsius.

29. The method of claim 25, wherein water of a temperature between 30 and 40 degrees Celsius is used to prepare the aqueous solution.

30. The method of claim 29, wherein the water temperature is temperature between 33 and 38 degrees Celsius.

31. The method of claim 29, wherein the water temperature is 35 degrees Celsius.

32. The method of claim 25, comprising mixing comprises 500 grams of sugar and 20 grams of the composition in 2 liters of water.

33. The method of claim 25 wherein the aqueous solution continuously produces CO₂ for at least 12 hours.
